(19) United States
(12) Patent Application Publication
(10) Pub. No.: US 2015/0174426 A1
(43) Pub. Date: Jun. 25, 2015

(54) UNDER-SHADOWING DISINTECTION SYSTEM

(71) Applicant: KONINKLIJKE PHILIPS N.V., EINDHOVEN (NL)

(72) Inventors: Mark David St. Germain, Dunstable, MA (US); Robert Alfred Feuersanger, Westford, MA (US); Zachary Dominguez, Carpinteria, CA (US); Lisette Manrique Miller, Taunton, MA (US)

(73) Assignee: KONINKLIJKE PHILIPS N.V., EINDHOVEN (NL)

(21) Appl. No.: 14/415,031

(22) PCT Filed: Jul. 9, 2013
(86) PCT No.: PCT/IB2013/055607

§ 371 Date: Jan. 15, 2015

Related U.S. Application Data
(60) Provisional application No. 61/673,432, filed on Jul. 19, 2012.
(60) Provisional application No. 61/740,721, filed on Dec. 21, 2012.

(51) Int. Cl. A61N 5/06; A61L 2/00; A61L 2/10

(52) U.S. Cl. CPC A61N 5/0624; A61L 2/10; A61L 2/0047; A61N 2005/0645; A61N 2005/0661

(57) ABSTRACT

A disinfection device includes a reflective surface (107) configured to direct light at a plurality of angles toward a target surface (108). The reflective surface forms a partial enclosure and has end portions (110). A light source (104) is disposed within the partial enclosure, wherein at least a portion of the light source is positioned within the partial enclosure such that light from the light source is reflected off the end portions to provide an angle of incidence on the target surface to treat an under-shadow region of an object or an uneven target surface.

### RELATED APPLICATION DATA

This disclosure claims priority to provisional application No. 61/673,432, filed on Jul. 19, 2012 and provisional application No. 61/740,721, filed on Dec. 21, 2012, both of which are incorporated herein by reference.

### BACKGROUND

1. Technical Field

This disclosure relates to disinfection instruments and more particularly to an under-shadowing radiation source and system for disinfecting regions shadowed by a medical instrument or having irregular or uneven surfaces.

2. Description of the Related Art

Infections in patients related to bacterial colonization on skin surfaces are well documented in the medical arts. Infection risk is greatest when medical devices penetrate the skin surface and create an ideal track for bacteria to migrate to sub-dermal tissue and vasculature. This can lead to serious risk of infection.

Known methods for achieving disinfection of skin underneath a catheter include using a disinfecting textile, which is inserted underneath the catheter. One disadvantage of using an opaque textile, gauze pad, or patch for this purpose is that it impairs visualization of the catheter's penetration site, and makes it difficult for clinicians to determine if an infection, or other symptoms of an infection (redness, etc.), are present at the penetration site.

The use of pulsed ultraviolet (UV) light energy may be employed to control skin level bacteria; however, UV exposure on skin comes with safety and efficacy issues. Uncontrolled exposure to UV light can be hazardous to human skin as well as represent an optical hazard to others from stray light. In addition, applying UV light as a means for disinfecting a penetration site of a medical device does not guarantee that portion of the medical device between the device and the skin surface is disinfected. The device acts as an obstruction which blocks collimated light from reaching the skin underneath it, potentially increasing risk of infection to the patient.

### SUMMARY

In accordance with the present principles, a disinfection device includes a reflective surface configured to direct light at a plurality of angles toward a target surface. The reflective surface forms a partial enclosure and has end portions. A light source is disposed within the partial enclosure, wherein at least a portion of the light source is positioned within the partial enclosure such that light from the light source is reflected off the end portions to provide an angle of incidence on the target surface to treat an under-shadow region of an object or an uneven target surface.

Another disinfection device includes a housing configured to form an internal cavity and a reflective surface formed in the internal cavity to direct light at a plurality of angles from the internal cavity. The reflective surface forms a partial enclosure and has end portions. An ultraviolet light source is disposed within the partial enclosure. The light source is positioned at least partially within the internal cavity such that light from the light source is reflected off the end portions to provide angled light to cover shadow regions caused by object obstruction or unevenness of a target surface.

A method for disinfecting obstructed surfaces includes providing a disinfection light system having a reflective surface configured to direct light from a light source at a plurality of angles toward a target surface to be treated, the reflective surface forming a partial enclosure and having end portions closest to the target surface; directing collimated light from the light source toward the target surface; and reflecting light off the reflective surface to provide angled light concurrently with the collimated light such that the angled light falls within an under-shadow region of the target surface.

These and other objects, features and advantages of the present disclosure will become apparent from the following detailed description of illustrative embodiments thereof, which is to be read in connection with the accompanying drawings.

### BRIEF DESCRIPTION OF DRAWINGS

This disclosure will present in detail the following description of preferred embodiments with reference to the following figures wherein:

FIG. 1 is a perspective view of a disinfection system in accordance with one illustrative embodiment;

FIG. 2 is a cross-sectional view of a disinfection system having a protective cover and showing treatment of an under-shadow region;

FIG. 3 is a schematic diagram of a disinfection system with spacers having motion of the system controlled by a mechanism and having an adjustable light source related to the reflective surface in accordance with another embodiment; and

FIG. 4 is a block/flow diagram showing a method for employing a disinfection system in accordance with illustrative embodiments.

### DETAILED DESCRIPTION OF EMBODIMENTS

In accordance with the present principles, systems and methods are described that provide for radiation in under-shadowing zones for sterilization in biological applications. In one embodiment, the present principles provide a light source (e.g., an ultraviolet light source) that creates wide, controlled illumination configured to produce shallow angled radiation delivered underneath a medical instrument or device (e.g., a catheter) or in between uneven portions of a surface to be treated. The angled radiation provides top side (under direct radiation) and bottom side (in the shadow) sterilization such that disinfection of a medical penetration site or other location may be achieved. For purposes of this disclosure, under-shadow, under-shadow regions, under-shadowing, etc. will refer to regions of shadow that would have been produced due to collimated light on an obstruction object or portion of a surface to be treated.

The controlled illumination source permits directional control of the radiation (UV energy) to ensure safety and/or efficacy for both the subject and others in the surrounding environment. The present embodiments may take the form of a hand-held instrument, a mechanically controlled instrument, a computer controlled instrument or other platform.

In one example, the present principles are employed at an insertion site of a medical device (e.g., a catheter, needle, guide wire, etc.) that penetrates the skin surface. At the penetration site, a catheter or other medical device includes a portion that is retained on the skin surface, and acts as an obstruction which blocks collimated light from reaching the skin underneath it. The present embodiments allow for shallow-angle light to be reflected underneath the obstruction (e.g., the catheter), to obtain disinfection in shadow areas, while maintaining collimated light output for treatment of the unobstructed skin surface. Use of light treatment for object under-shadowing permits disinfection under the object while maintaining visualization to the penetration site.

In accordance with a particularly useful embodiment, an under-shadowing disinfection device includes a reflector disposed in a fixture or light box. The reflector includes a geometrical relationship with a radiation source. Radiation source will be used hereinafter to include an electromagnetic radiation source (light, heat, etc.) although particle radiation sources (e-beam, etc.) may be employed as well in some embodiments. In one embodiment, a cylindrical light source is mounted at a defined location within the reflector to ensure a sufficiently wide angle relative to an instrument is achieved on the surface. The material of the reflector or coating of the reflector is selected to provide for optimal reflection of the radiation (e.g., UV light). The device enables shallow-angle light to reach the skin target underneath obstacles (under-shadowing) on the skin surface while concurrently ensuring light output is delivered to the skin surface that is free of obstacles. It should be understood that the present principles will be described in terms of an illustrative example having an obstructive object on a surface to be treated. However, the present embodiments are useful where the surface to be treated includes waves, ripples, uneven portions, creases, cracks or other topography that may produce shadows.

It should be further understood that the present invention will be described in terms of medical instruments; however, the teachings of the present invention are much broader and are applicable to instruments used with or in conjunction with a medical procedure. In some embodiments, the present principles are employed in disinfecting below any number of other objects, which may have application in the food industry, pharmaceutical industry or any other application where disinfecting is needed. In particular, the present principles are applicable to disinfecting tissue surfaces such as skin or other biological surfaces.

Reference in the specification to “one embodiment” or “an embodiment” of the present principles, as well as other variations thereof, means that a particular feature, structure, characteristic, and so forth described in connection with the embodiment is included in at least one embodiment of the present principles. Thus, the appearances of the phrase “in one embodiment” or “in an embodiment”, as well any other variations, appearing in various places throughout the specification are not necessarily all referring to the same embodiment.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended, as readily apparent by one of ordinary skill in this and related arts, for as many items listed.

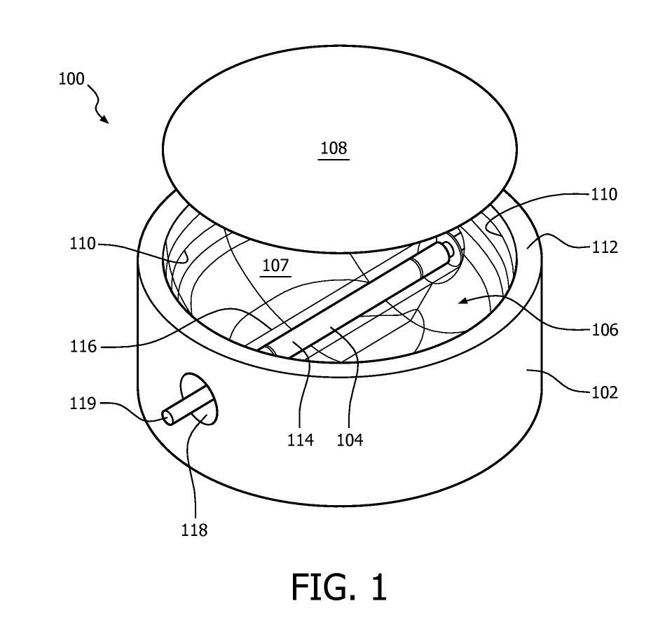

Referring now to the drawings in which like numerals represent the same or similar elements and initially to FIG. 1, an illustrative disinfection system 100 is shown in accordance with one embodiment. The system 100 includes a housing 102 configured to house a source 104, such as a bulb 114 or other radiation source. The source 104 may include a UV light although other sources may also be employed, e.g., an infrared source, etc. depending on the application. The housing 102 is configured to receive the source 104 and hold the source 104 in a fixed position although adjustments to the source 104 may be made. The position of the source 104 is configured to maintain a geometric relationship with a reflective surface 106 formed within the housing 102. The reflective surface 106 includes a parabolic, arcuate or cup-shaped portion 107 in a center region of the housing 102. Portion 107 provides collimated light, which is directed toward a target 108 (e.g., a skin surface) for direct disinfecting of a surface 108 and any instrument on the surface 108. Note that FIG. 1 shows a back portion of the target 108 so that the internal components of the system 100 are visible in perspective.

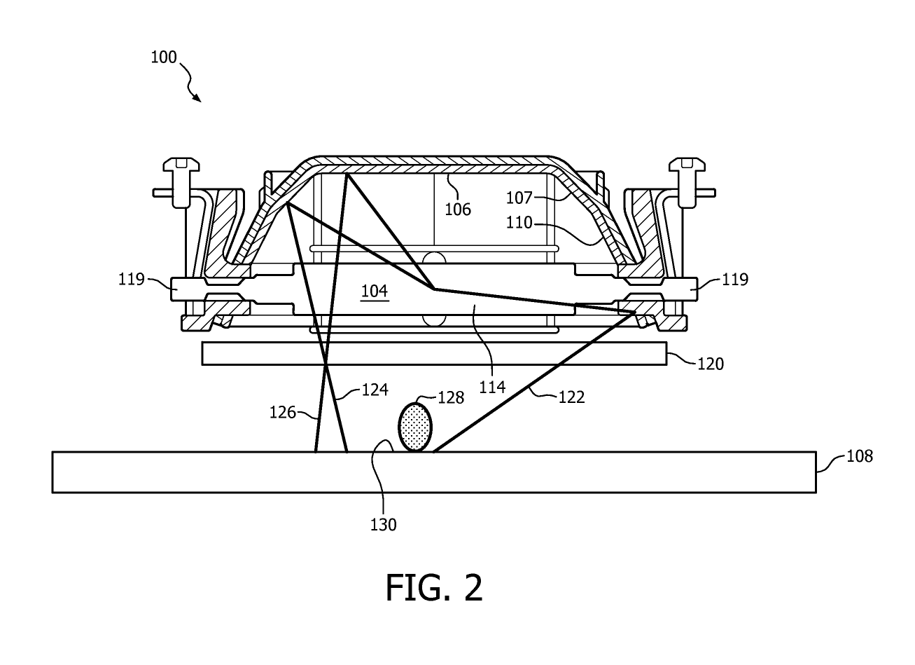

The reflective surface 106 further includes a portion 110 having a larger geometric gradient (steeper) around an outside portion of the reflective surface 106. The portion 110 is positioned a distance apart from the source 104 to permit light reflected from the portion 110 to be directed at a shallow angle relative to the target 108. In addition, the source 104 is set back a distance from an end surface 112 to provide the shallow angle and ensure that a sufficient amount of light from the source 104 is directed along the sides and bottom (in the shadow region) of any object (or uneven surface) on a surface of the target 108. This will be described in greater detail with reference to FIG. 2. The portion 110 may include ridges, fins or other structures disposed thereon to further support the reflection of light in accordance with the present principles. In addition, the portion 110 may include contoured surfaces to provide favorable redirection of light as needed for a particular application. In one embodiment, the contoured or structures surfaces of portions 110 may be actuated or adjusted to enhance control of the light angles.

The housing 102 may include a metal material, although polymeric or other materials may be employed. In one embodiment, the system 100 or parts thereof may include sterilizable materials. The reflective surface 106 may be removable from the housing 102 and replaced with other reflective surfaces configured for different applications or tools employed on the target surface 108. The reflective surface 106 is secured within or may be integrally formed within the housing 102. In the embodiment shown, the source 104 includes a cylindrical-shaped bulb 114 having a protective transparent casing 116 disposed about the bulb 114. The bulb 114 is placed and mounted within the housing 102 through an opening 118 formed through a sidewall of the housing 118. The bulb 114 is powered using electrodes 119 to provide, e.g., pulsed UV light energy during operation.

It should be understood that while the housing 102 is depicted as having a circular shape, other shapes may also be employed. These shapes may include an oval shape, a rectangular shape, a curved shape, an arcuate shape, etc. In addition, FIG. 1 depicts a cylindrical shaped bulb 114 disposed at or near a focal point or focal line for a shape of the reflective surface 106. Other shaped bulbs or multiple bulbs may be employed. For example, one or more point light sources may be employed, or combinations of light sources may be employed.

In one embodiment, position adjustment may be provided using adjustment mechanisms capable of moving the bulb 114 to control an amount and intensity of the light directed at the target 108. The relationship of the geometry of the reflector 106, and position of the bulb 114 relative to the reflector 106, controls a pattern of light and intensity delivered to the target 108 at a particular distance from the system 100. The geometry of the reflector 106 also enables the creation of simultaneous light output at shallow angles and as collimated light. Adjustments may be made to the reflector as well as the position of the source 104. For example, portion 110 may include fins or louvers which can be adjusted to provide different angles for reflected light.

Referring to FIG. 2, a cross-sectional view of the system 100 is shown in an operational position. The system 100 is held by hand or using a mechanical system (not shown) at a distance from the target 108. The target 108 may include a surface such as live tissue, e.g., skin sites, catheter insertion sites, intravenous (IV) sites, etc. On the target 108, a cross-sectional view of a medical instrument 128 is depicted. The instrument 128 may include a catheter, a guide wire, a probe, a needle, etc. The instrument 128 is disposed at or near the surface of target 108 and, as such causes, a shadow region 130. The shadow region 130 may also be caused by unevenness of the surface of the target 108 or other features that may cause under-shadowing or shadow areas.

During operation, the system 100 is powered to generate UV light pulses from the source 104. As described above, the bulb 114 radiates light which falls directly incident on the surface 108. The shadow region 130 is blocked from this direct light from the bulb 114. The cup-shaped reflective surface 106 is shown reflecting three rays 122, 124 and 126 to demonstrate the present principles. Ray 126 is reflected off a back portion of the reflective surface and, in addition to direct light, more or less provides collimated light on the target 108 and on a top portion of the instrument 128. Ray 124 is reflected off a portion of the reflective surface 106, which is curved such that the instrument 128 is located in a focal position relative to the geometry of the reflective surface 106. Ray 124 provides some coverage of the shadow region 130. Ray 122 is reflected off the steeper portion 110 of the reflective surface 106. The steeper portion 110 provides a shallow angle that penetrates deeply into the shadow region 130.

An optional protective cover 120 may be employed. The cover 120 permits the appropriate radiation to be transmitted therethrough and prevents access by a use to the bulb and internal surfaces of the system 100. It should be noted that if a protective cover is employed, the angles of light should account for refraction through the cover 120. In such a case, the reflector 106 may be designed or adjusted to account for the refraction angles through the cover 120.

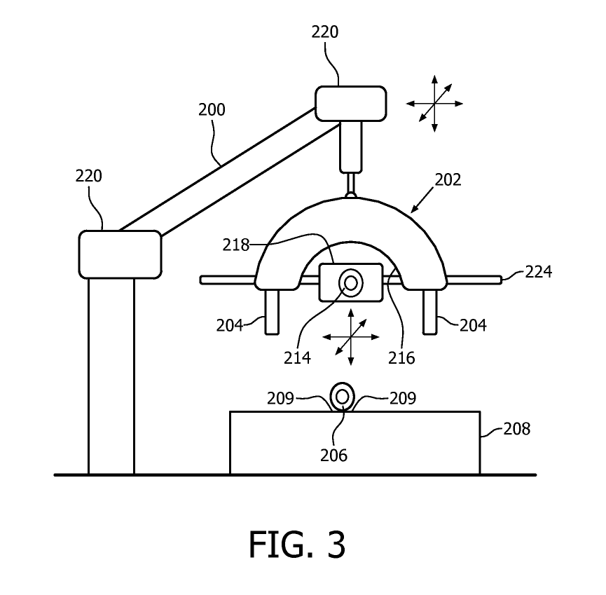

Referring to FIG. 3, another embodiment includes an illustrative mechanical linkage 200 for maneuvering and securing a disinfection system 202 (similar to system 100 of FIG. 1), shown in cross-section. The system 202 includes a source 214 disposed in a focal location of a curved reflective surface 216. The source 214 may be adjustable relative to the reflective surface 216 using an adjustment mechanism 218. The source 214 may be moved into/out of the plane of the page as well as up, down, left and right. The adjustment mechanism 218 may include devices such as set screws, slots, actuators, servos, etc.

In addition, the linkage 200 may be employed to move the system 202 to a position at or near a target area 208 to be disinfected. The linkage 200 may provide six degrees of freedom for maneuvering the system 202. The linkage 200 may be hand operated or may employ powered actuation devices, which may be controlled by computer or by manual controls. The linkage 200 may include locking mechanisms 220 to hold relative positions at joints of the linkage 200.

System 202 may include a spacer or spacers 204 that are configurable along with adjustments of the linkage 200 and/or the source 214 to provide optimized and homogenous light delivery to the target surface 208. The spacers 204 set a desired distance between the system 202 and the surface 208 to ensure the best results for disinfecting a particular object 206 and under-shadow surfaces 209 underlying that object 206. Varying the distances between the system 202 and the object 206 may provide a sweep of the area to provide even greater coverage of the target surface 208. A shield 224 may be provided at portions or completely around a circumference of the light to reduce reflected radiation exposure to surrounding regions and to protect others in the area.

Articulation of the linkage 200, the securing of the joints 220, movement of the spacers 204, the adjustment of the source 214, the adjustment of the reflector 216 and any other motions may be coordinated and controlled by hand or by computer. The system and methods described herein may be applied in optical applications where a controlled dispersion of light energy is desired, for example, in applications where UV curable epoxy resins are employed, e.g., in dental applications, or where epoxies are to be cured in the presence of an obstructing object. In medical applications, the present principles may be employed for applying UV light energy to surfaces for disinfection. Surfaces may include live tissue, such as, e.g., skin, instrument insertion sites, IV sites, etc. Surfaces may also include inorganic materials, such as, e.g., table tops, reusable medical devices, etc.

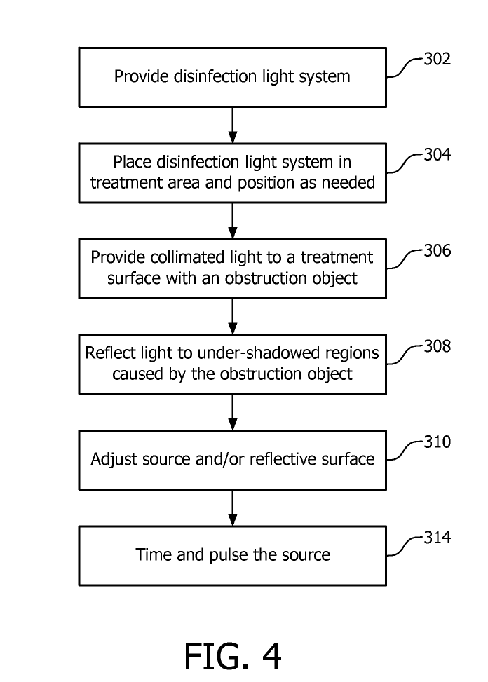

Referring to FIG. 4, a method for disinfecting an obstructed surface is shown in accordance with illustrative embodiments. The elements depicted in the FIG. 4 may be implemented in various combinations of hardware and software and provide functions which may be combined in a single element or multiple elements.

It should also be noted that, in some alternative implementations, the functions noted in the blocks may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

In block 302, a disinfection light system is provided that includes a reflective surface configured to direct light from a light source at a plurality of angles toward an opposing surface to be treated. The reflective surface forms a partial enclosure with end portions closest to an opposing surface to be treated (e.g., a skin surface) providing a steepest reflected angle for light. The reflective surface may be adjusted using fins or louvers or other actuated surfaces to change a reflected angle.

In block 304, the disinfection system is brought to the treatment area. This may include employing a position adjustment mechanism. This mechanism may be computer controlled or manually controlled. In another embodiment, the disinfection system is handheld. One or more spacers may be provided to define an offset distance between the end portions and the opposing surface. The spacers may be adjustable (manually or automatically) to alter the offset distance to the target surface. This helps to control the exposure area and provides the proper gap between the disinfection system and the surface.

In block 306, collimated light is directly propagated from the light source toward the object. In block 308, light reflected off the reflective surface provides angled light concurrently with the collimated light. The reflective surface is configured to provide the angled light such that the angled light falls within an under-shadow region of the object or obstruction on the opposing target surface. The angled light preferably includes a sharp or shallow angle to have a directional component that hits lateral sides of the object and the regions laterally adjacent to the object.

In block 310, adjustments to the source or reflective surface may be made. The angles provided for light rays are generated and controlled by providing a specific shape to the reflective surface. In one embodiment, the reflective surface includes an arcuate shape having a bottom surface continuing to the end portions, and the end portions include a steeper grade than other portions of the arcuate shape. It should be understood that the geometry of the reflective surface may be integrally formed as a single reflector or may include a plurality of pieces that can be moved to control the treated area. In one embodiment, the reflective surface may have movable portions, especially at the end portions (110) that permit adjustments to the shape of the reflective surface. The end portions of the reflective surface reflect a shallowest incoming angle to cover the under-shadow regions of the object. In addition, the position of the light source relative to the reflection surface permits the angled light to form an acute ray angle relative to the surface.

In another embodiment, a position of the light source is adjusted to control a treatment coverage area. It should be noted that the treatment area may be adjusted in accordance with a size and shape of the obstruction object. The treatment area may be asymmetrical, e.g., more radiation may be placed closer to an entry site than away from the entry site or the object or target surface may have an odd shape where radiation is more useful on one side of the object than the other, etc. The adjustment of the reflector and/or the light source may be computer controlled or manually controlled.

In one embodiment, the opposing surface may include a skin site, and the object includes a medical instrument. In such an embodiment, safety of the patient being treated and physicians, technicians etc. present in the immediate area is of concern and ameliorated by the present principles.

In block 314, the light source includes a UV light source. The light source may be pulsed to reduce that amount of incident energy. The UV light source may also be timed as controlled by a computer or other hardware device. The time of exposure may be maintained and recorded to ensure the least amount of skin exposure to UV light. In addition, the housing of the device is opaque to UV light, and the UV light exiting the surface may include a circumferential UV shield to prevent exposure of others in the environment.

The functions of the various elements shown in the FIG. 4 can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk-read only memory (CD-ROM), compact disk-read/write (CD-R/W), Blu-Ray™ and DVD.

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for under-shadowing disinfection systems and methods (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws, what is claimed and desired protected by Letters Patent is set forth in the appended claims.

1. A disinfection device, comprising:
a reflective surface configured to direct light at a plurality of angles toward a target surface, the reflective surface forming a partial enclosure and having a central portion intermediate portion and end portion having a steeper geometric gradient than the intermediate portion, said end portion being flared outwardly; and
a light source disposed within the partial enclosure, wherein at least a portion of the light source is positioned within the partial enclosure such that light from the light source is reflected off the end portion to provide a shallow angle of incidence on the target surface which allows the light to penetrate within an under shadow region of an object or an uneven target surface in order to treat such surfaces.

2. The device as recited in claim 1, wherein the target surface includes a skin site, and the object includes a medical instrument.

3. The device as recited in claim 1, wherein the central portion of the reflective surface includes an arcuate shape having a bottom surface continuing toward the end portion.

4. The device as recited in claim 1, wherein the reflective surface is shaped to concurrently provide collimated light and light that falls within the under-shadow region.

5. The device as recited in claim 1, wherein the light source includes an ultraviolet light source.

6. The device as recited in claim 1, further comprising an adjustment mechanism configured to adjust a position of the light source relative to the reflective surface.

7. The device as recited in claim 1, further comprising one or more spacers configured to provide an offset distance between the end portion and the target surface.

8. The device as recited in claim 1, further comprising a protective shield further enclosing the partial enclosure and configured to prevent access to the light source and reflective surface.

9. The device as recited in claim 1, further comprising an adjustment mechanism configured to adjust a configuration of the reflective surface.

10. A disinfection device, comprising:
a housing configured to form an internal cavity;
a reflective surface formed in the internal cavity to direct light at a plurality of angles from the internal cavity, the reflective surface forming a partial enclosure and having a central portion, intermediate portion and end portion having a steeper geometric gradient than the intermediate portion, said end portion being flared outwardly; and
an ultraviolet light source disposed within the partial enclosure, the light source being positioned at least partially within the internal cavity such that light from the light source is reflected off the end portion to provide shallow angled light which penetrates within shadow regions caused by object obstruction or unevenness of a target surface in order to cover such regions.

11. The device as recited in claim 10, wherein the target surface includes a skin site, and the object includes a medical instrument.

12. The device as recited in claim 10, wherein the central portion of the reflective surface includes an arcuate shape having a bottom surface continuing to the end portion.

13. The device as recited in claim 10, wherein the reflective surface is shaped to concurrently provide collimated light and light that falls within the shadow regions.

14. The device as recited in claim 10, further comprising an adjustment mechanism configured to adjust a position of the light source relative to the reflective surface.

15. (canceled)

16. (canceled)

17. (canceled)

18. (canceled)

19. A method for disinfecting obstructed surfaces, comprising:
providing a disinfection light system having a reflective surface configured to direct light from a light source at a plurality of angles toward a target surface to be treated, the reflective surface forming a partial enclosure and having a central portion, intermediate portion and end portion closest to the target surface, said end portion having a steeper geometric gradient than the intermediate portion, said end portion being flared outwardly;
directing collimated light from the light source toward the target surface; and
reflecting light off the reflective surface to provide angled light concurrently with the collimated light such that the angled light rejecting off the end portion falls within an under-shadow region of the target surface.

20. (canceled)

21. (canceled)

22. (canceled)

23. (canceled)

24. (canceled)

25. (canceled)
